(19) United States
(12) Patent Application Publication
(10) Pub. No.: US 2004/0064078 A1
(43) Pub. Date: Apr. 1, 2004

(54) Foot harness and stretching aid

(76) Inventor: Dara E. Winters, Belleview, NJ (US)

Correspondence Address: JOHN P. LUTHER, 25330 62ND AVENUE S., CC-303, KENT, WA, 98032, US

(21) Appl. No.: 10/331,052

(22) Filed: Dec. 27, 2002

Related U.S. Application Data
(60) Provisional application No. 60/414,859, filed on Sep. 30, 2002.

(51) Int. Cl. A61F 5/00

(52) U.S. Cl. 602/27

(57) ABSTRACT

The present invention provides a foot harness assembly to be worn about the foot and ankle of the user, and which comprises an ankle securing means, and a foot securing means, both of which provide a convenient hand-hold and/or foot-hold for a user during exercise, and/or any physical act or exertion and which are connected to each other by at least one member which also provides a convenient hand-hold and/or foot hold for a user during exercise motions and stretching; optionally, the connecting member and/or any of the ankle or foot securing means further comprises one or more hand holding and/or foot holding means as desired for virtually unlimited variability of use in any contemplated physical stretching or exertion exercise or act.

### RELATED APPLICATION

This application claims priority to U.S. provisional patent application Serial No.: 60,414,859, filed Sep. 30, 2002.

### FIELD OF THE INVENTION

The present invention relates to a method and apparatus for use in performing stretching exercises and various athletic acts, and particularly to a foot harness for use as an aid in the performance of stretching of muscles, tendons and ligaments, during flexion, extensions, exercising, athletics, yoga exercises, balance and coordination exercises, physical therapy, dance exercises, martial arts and the like. The invention further relates to a method of doing business encompassing the design, manufacture, marketing, sales and licensing of the inventive foot harness and various of its contemplated embodiments, and additionally to methods of instruction and teaching aids in use of the inventive foot harness.

### BACKGROUND OF THE INVENTION

Physical therapists, athletic trainers, yoga practitioners and the like have long recognized the advantages of a method and/or device providing assistance in stretching exercises. While many forms of exercise devices have been developed there currently exists a need for a simple and effective portable device which can be easily carried and fitted on and operated by a user to facilitate more easeful stretching and release of muscular contraction during physical exercises and exertions. Perhaps more importantly there has long existed a need for such a portable device as an easily carried and fitted foot harness apparatus to be worn during any type of physical exercise and exertion, and especially during an entire workday, to aid in muscle stretching and contraction with reduced fatigue and benefiting muscle toning, cardiovascular exercise, aerobic conditioning and the like.

Heretofore, several exercise devices and aids have been described, but none fulfilling the need satisfied by the instant inventive foot harness. For example, U.S. Pat. No. 6,402,669 describes a portable leg exercising apparatus said to provide for a reduced strenuous exercise, and which comprises a mat integrated with two rotatable posts having adjustable height heel cups, and which posts are adjustable in height. Rubber springs situated inside or outside the posts enable rotation. As described, a user lies down on a mat and begins to achieve a synergistic benefit of leg elevation while performing leg exercises. This device is not only cumbersome, but does not even approach the level of portability and ease of use of the instant inventive stretching device.

U.S. Pat. No. 5,647,827 describes a belt assembly exercise device to be worn around the torso, and which comprises a plurality of layers in which the first layer is formed of rigid plastic and a second layer is formed of a cushioning material. Further included in the belt assembly are upper and lower back support areas extending from the belt assembly and a plurality of attachment rings connected to the belt assembly with closure strips for removably attaching the belt assembly to the torso of the user. Further included in the device are hand assemblies with each assembly including finger holes for receiving the fingers of a user, and a hand strap for removably attaching each hand assembly to a hand of a user, and an elastic resistance member having attachment clips situated thereon for detachably connecting the hand assembly to one of the attachment rings on the belt assembly. Additionally a pair of anklet assemblies are provided with each such assembly including a ankle wrap for removably attaching each ankle assembly to an ankle of a user, a foot strap for removably attaching each ankle assembly to a foot of a user, a heel strap for extending around the bottom of the heel of a user and an elastic foot resistance member provided with attachment clips for detacably connecting the anklet assembly to one of the attachment clips on the belt assembly.

U.S. Pat. No. 5,137,272 describes a personal exercise device which has a back foundation plate or belt equipped with an extension pole and hand and leg elastic resistance components.

U.S. Pat. No. 5,433,688 describes a personal exercise device comprising a belt with elastic resistance hand grips and/or wrist cuffs which are attached to the front section of the belt, and which is exclusively designed for exercising the upper body of a user.

U.S. Pat. No. 6,283,897 discloses an ankle and hip exercising apparatus designed for exercising ankle and hip using dorsi ankle flexion, plantar ankle flexion, ankle rotation, and hip rotation. As described, a user may employ unidirectional resistance for strength and range of motion, development, or bi-directional resistance to develop proprioception. An overshoe equipped with a sole with pivots holds the foot in place, wherein the overshoe is mated with a shaft having a rounded top. Elastomeric resistance members supply the desired resistance. A further feature is a combination disc and hemispherical ball support which is employed to distribute weight thereby producing stress about the ankle in various foot areas. Various other foot exercising devices, along with lower extremity muscle conditioning devices, ankle exercising devices and hip exercising devices are described in this reference, one of which is U.S. Pat. No. 5,100,129 describing a lower leg exercise device including a foot harness equipped with an elasometric resistance member in the form of a length of surgical tubing. The tubing is said to extend from the foot harness to a piece of furniture.

As a further example of conventional art, U.S. Pat. No. 5,489,251 describes an exercise device provided in the form of a foot harness which is securable about the foot of a user and further equipped with a thigh harness having a rigid plate member securable about the thigh of a user, and a tension adjusting mechanism connected between the foot harness and thigh harness. A tension adjusting mechanism is also employed in this device, which includes a tubular housing having an elongated passageway therein which terminates in a housing, and which further includes a spring plate with spring, a flexible tension member and a positioning member connected to the spring plate. The described assembly is said to allow the user to selectively position the spring plate in a desired position within the passageway, all of which provides for variable resistance loading for member specific exercises, such as calf muscle and the like.

Another example of a body harness and foot harness assembly is described in U.S. Pat. No. 6,325,023 which is designed to enable a person to assist another disabled person in standing and walking exercises.

As can be seen, there exists neither in description or conventional product, a device and/or method comparable to the instant inventive foot harness which approaches in any way its ease and convenience of construction, adaptability, deployability and portability as well as its performance of exceptional ease of assistance in performing virtually any desired stretching and/or tensioning exercise or athletic act, yoga exercise, physical conditioning, physical therapy regimen and the like. The long awaited need for a portable easily carried and deployable device which is easily carried and fitted on and operated by a user to facilitate ease of stretching and release of muscular contraction during virtually any physical exertion due to any means is now provided by the instant inventive foot harness and method of use.

Additionally, an innovative method of business operation is provided by way of design, manufacture, marketing, sales and licensing of the inventive foot harness, all of which present advantageous business applications and opportunities which heretofore have not been available thereby providing new and unique industries.

The present inventive foot harness is more fully described, with preferred embodiments discussed in particular, in the following Summary of the Invention, discussion of Detailed Description, and Description of Drawings and Claims. It is to be understood, however, that the foregoing and following Detailed Discussion, et al. is intended to depict and/or provide preferred illustrations only, with a multitude of embodiments of the inventive foot harness, such as wrist and knee harnesses embodiments, method of use and business deployments and engagements for use therewith contemplated.

### SUMMARY OF THE INVENTION

In the broadest sense, the present invention provides a foot harness assembly to be worn about the foot and ankle of the user, and which comprises an ankle securing means, and a foot securing means, both of which provide a convenient hand-hold and/or foot-hold for a user during exercise, and/or any physical act or exertion and which are connected to each other by at least one member which also provides a convenient hand-hold and/or foot hold for a user during exercise motions and stretching. Optionally, the connecting member and/or any of the ankle or foot securing means further comprises one or more hand holding and/or foot holding means as desired for virtually unlimited variability of use in any contemplated physical stretching or exertion exercise or act. The invention is more fully described by the following Detailed Description of Preferred Embodiments with accompanying drawings.

### BRIEF DESCRIPTION OF THE DRAWINGS

FIG. 1 is a top perspective view of the foot harness apparatus according to a preferred embodiment of the present invention as fitted about a user's foot.

FIG. 2 is a top and side perspective view of the foot harness of the present invention as shown in FIG. 1.

FIG. 3 is a top perspective view of the foot harness apparatus according to a preferred embodiment of the present invention.

FIG. 4 is a top and side perspective view of the foot harness of the present invention as shown in FIG. 3.

FIGS. 5-7 illustrates various physical exercises as performed in conjunction with a preferred embodiment of the present inventive foot harness apparatus and assembly.

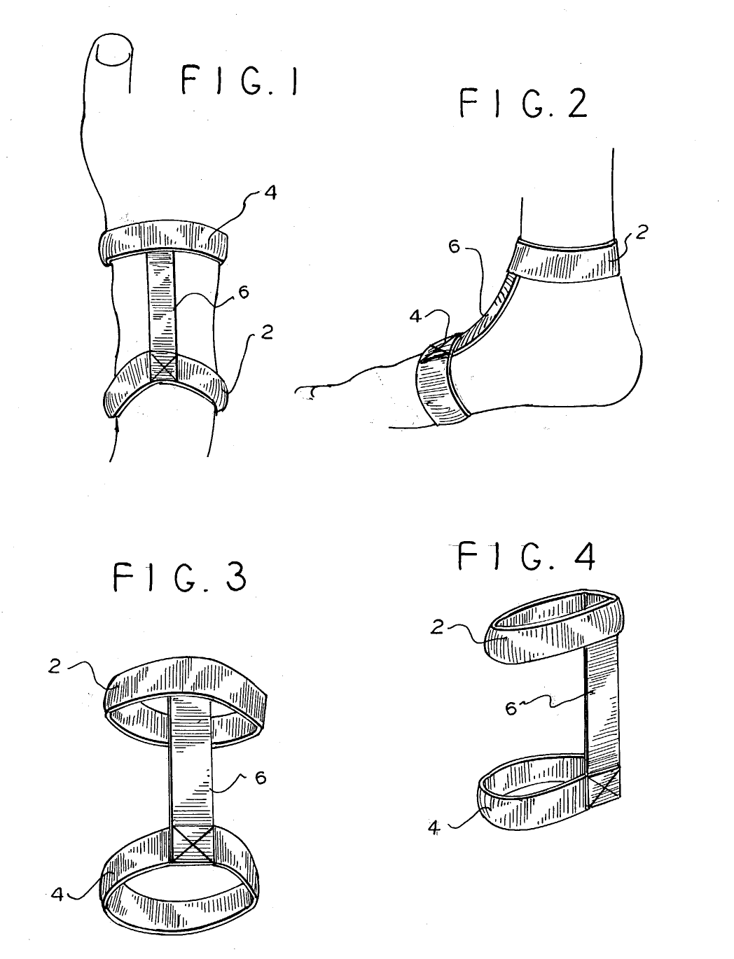

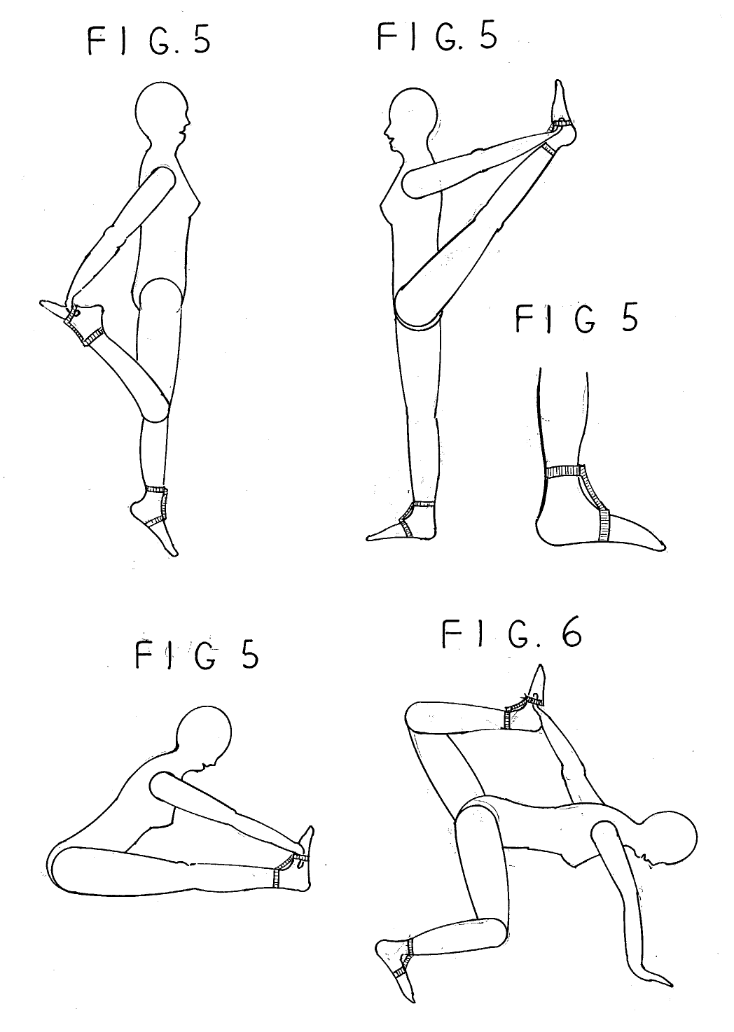

### DETAILED DESCRIPTION OF PREFERRED EMBODIMENTS

A preferred embodiment of the present invention is best illustrated in FIG. 1. The foot harness, apparatus and assembly comprises an ankle securing means 2, which is fitted and secured about the periphery of a portion of a user's ankle, and a foot securing means 4 which is fitted and secured about the periphery of a portion of a user's foot, preferably about a portion of the arch area. As shown, the ankle securing means 2 and foot securing means 4 are connected to one another by one or more longitudinal transverse hand holdable and/or foot holdable member 6, which transverses a portion of the dorsal/anterior aspect of the user's foot when developed thereon. Ankle securing means 2 and foot securing means 4 are preferably straps, and either one or both may be composed of materials possessing elastic qualities and which can be easily deployed on and off the user's ankle and foot portions, and distanced apart by the length of member 6, which length may vary as desired depending upon such factors as the particular user's anatomy and physical exertions contemplated. According to the materials used for either ankle securing means 2 and/or for foot securing means 4 the desired degree of stretch and/or resistance imparted to each means may be conveniently achieved, depending on a user's motion development. Either one of ankle securing means 2 and/or foot securing means 4 are continuous straps or may be provided with any type of clasp, fastening material or securing means, such as Velcro or Velcro/elastic strap, to provide straps which encircle and close about the user's ankle and foot portions, respectively, preferably in a relatively tight fitting mode. The straps 2 and 4 may be made of any type of suitable material, such as canvas, nylon, cloth, rubber, or other fabric and/or fabric/elastic materials and the like.

Member 6 is of any type of suitable fabric material, and may be composed of a elastomeric resistance material which will stretch and contract according to the range of the user's motion development. Some preferred materials may be canvas, nylon and other fabrics or elastomeric resistance materials such as surgical tubing or cloth encased elastomeric material. In other preferred embodiments (not shown) more than one and a plurality of members 6 may be employed to vary bi-directional resistance as desired, and ease of stretching and physical exertion in the user's ability to secure a hand hold and/or foothold to member 6.

The inventive foot harness enables the user to achieve a further, more easeful stretch by assisting in proper stretching technique and aiding in muscular contraction. The foot harness also reduces fatigue of the all-day user during the workday by providing an added means of muscular or muscloskeletal support.

Referring now to FIGS. 5-7, there are shown various physical stretching exercises being performed by a user in conjunction with a preferred embodiment of the present inventive foot harness and assembly depicted in FIG. 1. As illustrated in FIG. 5, for example, the foot harness provides a convenient and secure hand hold (or foothold) for essentially limitless degrees of freedom of the user's motion development during stretching exercises, allowing for a higher confidence level than would otherwise be obtainable in its absence, and concomitantly allowing for a higher degree of efficiency in such exercises.

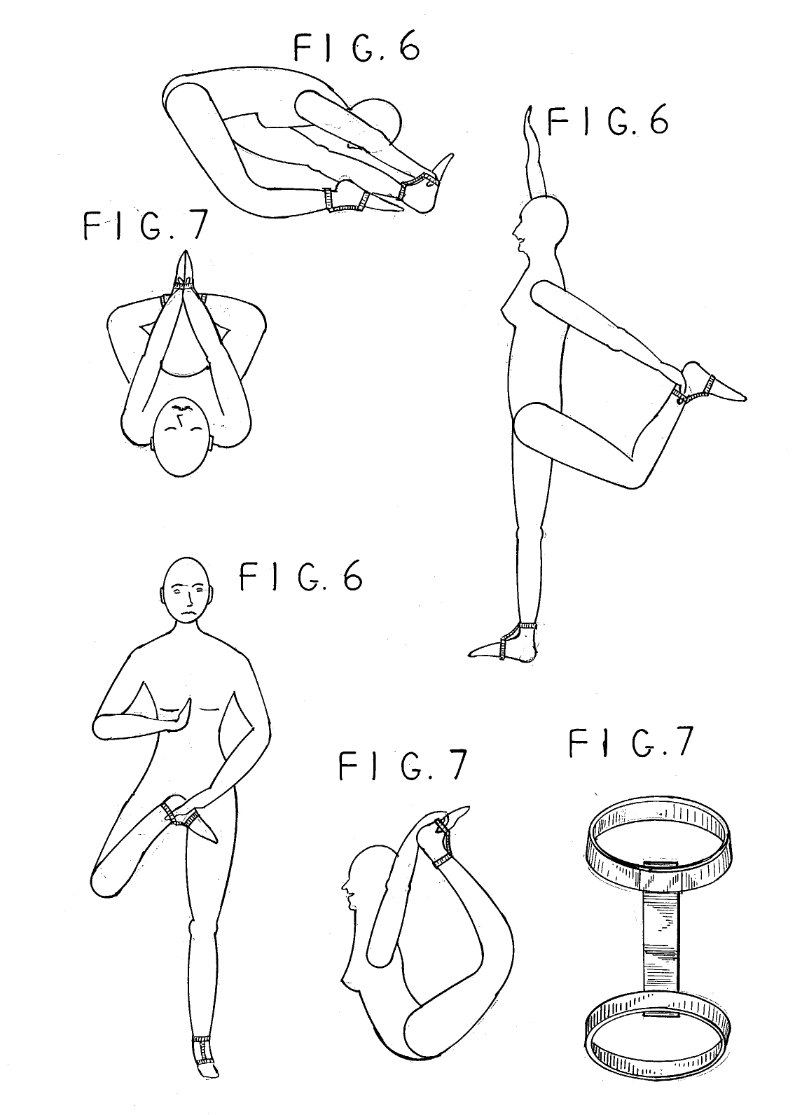

As shown for example, in FIGS. 6-7, all of the components of the inventive harness provide for convenient and secure hand holds and/or foot holds during virtually any type of physical stretching exercise or exertion contemplated by the user.

Further hand hold and/or foot hold means may be situated on and/or adapted to member 6 or any of ankle securing means 2 and/or foot securing means 4, as desired, or otherwise contemplated for any permutation or execution of usage.

Without desiring to limit the present invention in any way, some examples of stretching exercises advantageously benefiting from the aid of the deployed inventive foot harness are now discussed.

As is well known, human movement is more enjoyable and beneficial to exercise and other physical acts and extensions when the body is flexible and capable, and more importantly able, of performance without restriction or hindrance. To this end the present inventive foot harness enables ease of superior execution of the following selected exemplified stretching exercises.

As a particularly illustrative example, muscles associated with hip joints which flex and extend, and which are part of the lower back and knee, as well as various other muscles are easily stretched with the inventive foot harness. Some contemplated exercises include a bent knee hamstring stretch aided by the foot holds and/or hand holds of the inventive foot harness, as well as a bent knee inner hamstring stretch, a bent knee outer hamstring stretch all performed while the user is in a supine position, or a seated bent knee hamstring stretch. Further illustrative examples include a straight leg hamstring stretch, a straight leg inner hamstring stretch, a straight leg outer hamstring stretch, all from a supine position, or seated straight leg hamstring stretch. Further contemplated, for example, are the standing hamstring stretch, the side lying psoas stretch, the side lying rectus femorũs stretch, the standing rectus femorũs stretch, the prone hip external rotation stretch, the supine hip external rotator stretch, the seated hip external rotator stretch, the prone hip internal rotator stretch, seated hip internal rotator stretch, supine hip internal rotator stretch, hip abductor stretch, groin hip adductor stretch, seated hip adductor stretch, hip fexion, lateral gluteus maximus stretch, piriformis rotator stretch, oblique lateral trunk flexion, dorsal ankle stretch, soleus stretch, achilles tendon stetch, gastrocnemius (calf) stretch, outer gastrocnemius stretch, inner gastrocnemius stretch, evertor stretch, invertor stretch, foot pronator stretch, foot supinator stretch, metatarsal arch stretch, and various assisted stretching exercises.

A detailed discussion of various stretching executions can be found in “Active Isolated Stretching,” Aaron L. Mattes, Copyright© 1995 Aaron L. Mattes, the entirety of which is incorporated herein by reference.

Again, without intending to limit the present invention or the claims in any way, it is contemplated that virtually every human body muscle is advantageously exercised in stretching executions in conjunction with the aid of the present inventive foot harness. Included examples are the muscles deltoid, pectoralis major, coracobrachiolis, biceps, brachii, brachiolis, brachioradialis, gluteus medius, tensor fasciae, tensor fasciae latae, pectineus, rectus femoris, iliotibial band, tibialis anterior, gastrocnemius, ext. digitorum longus, tendon of ext. hallucis, peroneus longus, tibialis anterior, vastus medialis, vastus lateralis, sartorius, gracilis, adductor longus, iliopsoas, rectus abdominis, external oblique, serratus anterior, latissimus dorsi, teres major, sternocleidomastoid, infraspinatus, teres minor, latissimus dorsi, carpi flexor ulnoris, adductor magnus, tensor fasciae latae, semitendinosus, iliotibial tract, plantoris, soleus, trapezius, triceps brachii, gluteus maxiums, semimembranosus, gracilis, semitendinosus, biceps femoris, achilles tendon.

A detailed discussion of the aforementioned muscles and musculoskeletal anatomy in particular as benefited from use of the inventive foot harness and embodiments thereof can be found in “Illustrated Essentials of Musculoskeletal Anatomy,” 3^{rd} Edition, Sieg and Adams, Copyright© 1996, Megabooks, Inc., of which the entirety thereof is incorporated by reference herein. See also, for example, Greys Anatomy, a well known treatise on human anatomy with detailed discussions of the musculoskeletal archipelago, the entirety of which is incorporated herein by reference.

A further example of beneficial application of the inventive foot harness and its embodiments is in the practice of yoga. Particularly illustrative examples include such positions as Dhanur\overline{a}sana, or bow pose; D\overline{a}nusirsh\overline{a}sana, or head-to-knee pose; Paschimoth\overline{a}n\overline{a}, or posterior-stretching or forward-bending pose; Padona Sarv\overline{a}ng\overline{a}sana, or lotus shoulder stand; intermediate Matsy\overline{a}sana, Pavanamuktasana, or gas-relieving pose/wind-elimination pose; Pada Mayur\overline{a}sana, or lotus peacock pose; Arddha Matsyendr\overline{a}sana, or half spinal twist; Trikon\overline{a}sana, or triangular pose; Padahasth\overline{a}sana, or hand-to-feet pose; yoga mudra or yogic seal; \overline{A}karshna Dhanur\overline{a}sana, or shooting-bow pose; Baddha J\overline{a}nushirsh\overline{a}sana, or bound head-to-knee pose; Baddha Padm\overline{a}sana or bound lotus pose; Baddha Yoga Mudra or bound yogic seal; Eka. P\overline{a}da trikon\overline{a}sana, or single-leg triangle pose; Eharb\overline{a}sana, or child-in-womb pose; Kukkut\overline{a}sana, or lock pose; Natar\overline{a}j\overline{a}sana, or Kimn Dancer pose; Omk\overline{a}r\overline{a}sana, or “Om.” pose; Padhangush\overline{a}tsana, or tiptoes pose, Paschima N\overline{a}u\overline{a}sana, or forward boat pose; Poorna Matsy\overline{a}sana, or full Ash pose; Poorna Matsyendr\overline{a}sana, or full spinai twist; Sasang\overline{a}sana, or Rabbit pose; Tolangul\overline{a}sana, or Seale pose; Uttlitha Dhanur\overline{a}sana, or raised-bow pose; Utthitha Paschimoth\overline{a}n\overline{a}sana, or lifted forward bending pose; Vakr\overline{a}sana, or Crooked pose; Yoga Nidr\overline{a}sana, or Yogic sleep; Mah\overline{a}Mudra, or Great Seal; and Sthala, or Ground basti.

It is furthermore contemplated that the foot harness of the present invention be adapted for use as a hand harness, wrist harness and knee harness, inclusive of all the aforementioned essential features of the foot harness equipped with all essential hand and/or footholds as described, and useful in any physical exercise exertion, yoga position, physical therapy act and the like.

Additional contemplated uses include physical therapy applications, floor exercises, any meditational exercise, sensual or sexual exercises or positions and virtually any physical contortion of the body through physical exertion, with essentially unlimited degrees of freedom of a user's motion development, thereby enabling full performance without restriction or hindrance from natural or genetic ability, or otherwise natural range of performance.

1. A physical exertion aid to be worn about the foot and ankle of the user, comprising an ankle securing means, a foot securing means, hand and/or foot holding means situated on or about or contiguous to said ankle and/or foot securing means, wherein said ankle and foot securing means are connected to each other by a hand hold and/or foot hold connecting member means, which is optionally equipped with one or more additional hand and/or foot holding means.

2. The physical exertion aid of claim 1, which is a foot harness apparatus, and wherein said ankle securing means is fitted and secured about the periphery of a portion of a user's ankle, and wherein said foot securing means is fitted and secured about the periphery of a portion of a user's foot, and wherein said connection member means is present as one or more longitudinal transverse hand holdable and/or foot holdable strap means.

3. A process for the manufacture of the physical exertion aid of claim 1, comprising production of said hand and foot securing means and connecting each other with connecting member means.

4. A method of use of the physical exertion aid of claim 1 comprising its use in conjunction with performance of any physical stretching exercises, performance of yoga exercise, performance of physical therapy, performance of martial arts, performance of sexual acts and/or performance of any physical exertion involving any muscles of the human body.

5. A method of doing business comprising the manufacture of the physical exertion aid of claim 1, and further comprising the marketing, sales, and licensing of said physical exertion aid.

6. The physical exertion aid of claim 1, wherein said hand hold and/or foot hold connecting member comprises one or more longitudinal transverse hand holdable and/or foot holdable members which preferably transverse a portion of the dorsal/anterior aspect of a user's foot.

7. The physical exertion aid of claim 1 wherein said ankle securing means and/or said foot securing means is a strap means, optionally composed of materials possessing elastic qualities or non-elastic qualities, and which are distanced apart by the length of said connecting member.

8. The physical exertion aid of claim 1 wherein either said ankle securing means or said foot securing means are continuous strap means or are equipped with fastening means to encircle and enclose said ankle securing means and/or said foot securing means about a portion of a user's ankle or foot, respectively.

9. The physical exertion aid of claim 1 wherein any of said ankle securing means, foot securing means and connecting member comprise one or more additional hand holdable and/or foot holdable means.

10. A physical exertion aid to be worn about the hand and wrist of the user comprising a hand securing means, a wrist securing means, hand and/or foot holding means situated on or about or contiguous to said hand securing means and/or wrist securing means, and wherein said hand and wrist securing means are connected to each other and distanced apart by on or more hand hold and/or foot hold connecting member, which traverses any portion of a user's wrist portion.

11. A physical exertion aid to be worn about the leg portions contiguous to a knee of the user comprising first and second leg securing means situated on leg portions divided by a knee portion, further comprising hand and/or foot holding means situated on or about or contiguous to said first and second leg securing means, and wherein said first and second leg securing means are connected to each other and distanced apart by a handhold and/or foot hold connecting member which traverses any portion of a user's knee portion.

12. A process for the manufacture of the physical exertion aid of claim 10 comprising production of said hand and wrist securing means and connecting each other with said connecting member.

13. A method of use of the physical exertion aid of claim 10 comprising its use in conjunction with performance of any physical stretching exercises, performance of yoga exercises, performance of physical therapy, performance of martial arts, performance of sexual acts and/or performance of any physical exertion involving any muscle(s) of the human body.

14. A method of doing business comprising the manufacture of the physical exertion aid of claim 10, and further comprising the marketing, sales and licensing of said physical exertion aid.

15. A process for the manufacture of physical exertion aid of claim 11 comprising production of said hand and wrist securing means and connecting each other with said connecting member.

16. A method of use of the physical exertion aid of claim 11 comprising its use in conjunction with performance of any physical stretching exercises, performance of yoga exercises, performance of physical therapy, performance of martial arts, performance of sexual acts and/or performance of any physical exertion involving any muscle(s) of the human body.

17. A method of doing business comprising the manufacture of the physical exertion aid of claim 11, and further comprising the marketing, sales, and licensing of said physical exertion aid.
